(19) United States
(12) Patent Application Publication
(10) Pub. No.: US 2011/0144484 A1
(43) Pub. Date: Jun. 16, 2011

(54) METHOD AND DEVICE FOR MONITORING A MEDICAL PROCEDURE SUCH AS A VASCULAR SURGERY OPERATION

(76) Inventors: Sebastian Schmidt, Weisendorf (DE); Anke Weissenborn, Weil am Rhein (DE)

(21) Appl. No.: 12/964,868

(22) Filed: Dec. 10, 2010

(30) Foreign Application Priority Data
Dec. 11, 2009 (DE) 10 2009 057 924.9

(51) Int. Cl. A61B 5/05

(52) U.S. Cl. 600/431

(57) ABSTRACT

In a monitoring method and device for monitoring a medical procedure, in particular a vascular surgery operation, at least one monitoring measurement in a monitoring region is acquired by a medical imaging device, and the at least one monitoring measurement takes place essentially simultaneously with the medical procedure.

### BACKGROUND OF THE INVENTION

1. Field of the Invention

The present invention concerns a method and a device for monitoring a medical procedure, such as a vascular surgery operation.

2. Description of the Prior Art

In vascular surgery operations, a danger exists that an anatomical area that is supplied with blood, and thus with oxygen, dependent on a vessel to be operated on (for example an artery) may be damaged, in particular by an undersupply and/or deposited particles. For example, given an operation on the cervical artery an undersupply of (and therefore damage to) the brain can occur.

For vascular surgery procedures, a perfusion of the vessel to be operated on is suppressed; the vessel is subsequently opened and a rerouting for the perfusion is put in place, for example a plastic tube. The vessel is subsequently operated on and (insofar as it is possible) sealed. After the operation the perfusion of the vessel is suppressed again and the rerouting for the perfusion is removed. The entrance point of the tube is subsequently sealed and the vessel is reopened for a perfusion.

A blocking of the vessel that lasts too long during the operation can lead to damage to an organ supplied by the vessel (or to the brain), such as a possible embolism due to deposited particles, for example from portions of the vessel wall and/or microthromboses and/or a coagulation (in particular an extravascular blood clot) at the tube wall, etc. Air embolisms and/or cerebral hemorrhages due to the blood thinning agents used during the operation can also be a cause of damage to the organ and/or the brain. In order to reduce and/or to avoid possible damage to an organ and/or to the brain, it is sought to block the vessel for as brief an amount of time as possible.

In a known monitoring method for monitoring a medical procedure, in particular a vascular surgery operation, a monitoring measurement ensues by means of an imaging device, but only after the vascular surgery operation. Possible damage to an organ and/or the brain thus can be established only after the operation. Countermeasures and/or reactions to damage that have already occurred can then take place too late, such that a danger of irreversible damage additionally exists.

### SUMMARY OF THE INVENTION

An object of the present invention is to provide a monitoring method that enables an immediate detection of possible danger to the patient during the medical procedure (in particular the vascular surgery operation) and therefore at least reduces and/or prevents the risk of damage to an organ and/or the brain.

The invention is a monitoring method for monitoring a medical procedure (in particular a vascular surgery operation), wherein at least one monitoring measurement in a monitoring region is acquired by means of a medical imaging device.

In accordance with the invention, the at least one monitoring measurement essentially ensues simultaneously with the medical procedure. Possible danger to the patient (in particular the danger of damage to an organ and/or the brain and/or other body parts) thus can be detected immediately upon the appearance of the danger in an image and can particularly advantageously be communicated to the personnel (in particular a physician) conducting the procedure. The danger of damage to an organ and/or the brain and/or other body parts thus can be at least reduced if not prevented before irreversible damage, by the administration of a countermeasure to prevent and/or reduce the damage by the physician insofar as necessary.

As used herein a monitoring region is a region that is located spatially separate from an operation region for the vascular surgery operation and is in particular located in the organ and/or brain and/or additional body part that is at risk. Furthermore, as used herein a medical imaging device is a computed tomography device, or particularly advantageously a magnetic resonance tomography device, such that a nearly immediate or only slightly delayed monitoring (substantially real-time monitoring) of the monitoring region is possible, for example, and a current potential danger to the patient thus can always be almost immediately detected and recognized. A supply—in particular a blood supply—and/or a degree of a supply of the organ and/or brain and/or additional body part is advantageously determined by means of the monitoring measurement, such that a possible undersupply and/or a possible incorrect supply can be detected immediately. Further disruptions—in particular air embolisms and/or deposited particles etc.—caused in the monitoring region by the vascular surgery operation should also additionally be detected by means of the monitoring measurement. As used herein a vascular surgery operation is a surgical operation on a blood vessel.

In an embodiment of the invention, multiple monitoring measurements ensue essentially continuously in succession. An essentially uninterrupted or continuous supply flow of the organ and/or brain and/or additional body part that is dependent on the vessel to be operated then can be advantageously achieved and a danger of damage can be detected immediately. The multiple monitoring measurements advantageously all take place essentially in parallel with the medical procedure, such that the organ and/or brain and/or additional body part can be monitored over a longer time period of the vascular surgery operation, and particularly advantageously can be monitored during the entire vascular surgery operation.

In another embodiment, at least one comparison measurement is acquired to detect at least one reference variable, a change in the monitoring measurement relative to the reference variable can be particularly advantageously detected. The comparison measurement is advantageously conducted at an area of the organ and/or brain and/or additional body part that is protected from a possible damage, or is conducted before the vascular surgery operation, such that a change in the monitoring measurement that is caused by the vascular surgery operation can be effectively detected. As used herein a reference variable is a variable that reflects a supply of the undamaged organ and/or brain and/or additional body part, for example a perfusion variable.

In a further embodiment of the invention, the at least one comparison measurement is acquired before the monitoring measurement and/or the medical procedure. In this context, “before” should be understood from a temporal standpoint. In this embodiment, a reference variable can advantageously be obtained that reflects a normal supply of the organ and/or brain and/or additional body part, such that changes in the supply of the organ and/or additional body part can be detected immediately during the vascular surgery operation.

Furthermore, the at least one comparison measurement can be acquired essentially in parallel with the monitoring measurement and/or the medical procedure. “Parallel” should also be understood from a temporal standpoint. In particular, the comparison measurement can take place together with the monitoring measurement in one measurement step, for example with both hemispheres of the brain being detected by a measurement.

When at least one monitoring variable obtained from the monitoring measurement is compared with the reference variable, a change in the supply of the organ and/or brain and/or additional body part of the examination region can be effectively detected. A comparison of the monitoring variable obtained from the monitoring measurement with the reference variable can ensue manually (performed by a physician, for example) or can particularly advantageously ensue via the medical imaging device (which has an evaluation unit for this purpose), for example. A comparison of the monitoring variable with the reference variable advantageously ensues immediately after the detection of the monitoring variable, such that a possible danger can be corrected before an occurrence of possible irreversible damage.

In another embodiment a warning signal is emitted given a change of the monitoring variable (obtained from the monitoring measurement) that lies outside of a tolerance range relative to the reference variable. A physician conducting the surgical operation can hereby be informed of a change to the supply (for example an undersupply) of the organ and/or the brain and/or additional body part immediately after appearance of the change. A diagnosis of the change by the physician advantageously ensues so that said physician can immediately introduce countermeasures with regard to the change in the supply. A tolerance range in this context is a range that represents a maximum, still-tolerable deviation of the monitoring variable obtained from the monitoring measurement relative to the reference variable. The output of the warning signal can take place optically and/or acoustically.

The monitoring measurement advantageously ensues by means of a perfusion imaging and/or an angiography imaging and/or a diffusion imaging and/or a susceptibility-weighted and/or T2*-weighted imaging. In addition to this, an image contrast of individual measurement methods of the imaging of the monitoring method can be additionally intensified via the addition of a contrast agent before the monitoring measurement. The contrast agent administration advantageously ensues by means of an injection of the contrast agent.

Furthermore, the invention encompasses a monitoring device for monitoring a medical procedure (in particular a vascular surgery operation) with a medical imaging device that is designed for working at least one monitoring measurement in a monitoring region.

In accordance with the invention device, the at least one monitoring measurement by means of the medical imaging device takes place essentially simultaneously with the medical procedure. A possible danger to the patient, in particular a danger of a damage to an organ and/or the brain and/or additional body parts, can hereby be detected immediately after an appearance of the danger and can particularly advantageously be communicated to a personnel (in particular a physician) conducting the procedure. The danger of a damage to the organ and/or brain and/or additional body part can therefore also be at least reduced and/or prevented (in particular before irreversible damage) in that a countermeasure to prevent and/or reduce the damage can be introduced by the physician insofar as it is necessary. The medical imaging device can be particularly advantageously formed by a mobile (in particular movable) imaging device so that it can be used for different monitoring regions of patients in a vascular surgery operation.

The monitoring device advantageously has a computer that is configured to make a comparison of at least one monitoring variable of the monitoring measurement with at least one reference variable of a comparison measurement, whereby a time-saving data evaluation can advantageously be achieved within the monitoring device. This additionally enables a time-saving detection of a possible danger of damage to the organ and/or brain. What should hereby be understood by a computer is in particular a unit that advantageously possesses a processor for a data evaluation and/or for controlling and/or regulating individual components of the monitoring device. In addition, the computer can have additional components, for example a storage element. As used herein, configured means specially equipped and/or specially designed and/or specially programmed.

The monitoring device can have an alarm output unit that is designed to output a warning signal given the presence of a deviation of the monitoring variable relative to a reference variable. A physician conducting the surgical operation thus can be notified of an endangerment of the organ and/or brain and/or body part (for example due to an undersupply) immediately after appearance of the change. A diagnosis of the change of the supply by the physician advantageously takes place, such that said physician can immediately introduce countermeasures relative to the change of the supply.

The monitoring device particularly advantageously includes a contrast agent unit that is designed to administer a contrast agent. The contrast agent can be supplied to intensify a contrast in the imaging before the monitoring measurement and/or a comparison measurement. The contrast agent unit can additionally be designed such that the contrast agent is supplied in stages given continuous monitoring measurements so that an essentially equally high contrast agent concentration and/or image quality can be achieved during the continuous monitoring measurements. The administration of the contrast agent advantageously ensues via an injection of said contrast agent. Furthermore, the contrast agent unit can be designed such that it injects a dose of contrast agent during the medical procedure, wherein the injection can be triggered by an action of the physician or can take place at pre-established intervals, and a measurement is triggered at a defined time interval after the injection. The time interval is hereby advantageously adapted to a time period that the injected contrast agent requires to reach the target organ.

The medical imaging device is particularly advantageously formed by a computed tomography device or particularly preferably formed by a magnetic resonance tomography device since these enable a particularly fast imaging with a high resolution of the monitoring region.

### BRIEF DESCRIPTION OF THE DRAWINGS

FIG. 1 illustrates a monitoring method according to the invention.

FIG. 2 illustrates a monitoring device according to the invention with a magnetic resonance tomography device in a schematic representation.

FIG. 3 illustrates the monitoring device with a computed tomography device in a schematic representation.

### DESCRIPTION OF THE PREFERRED EMBODIMENTS

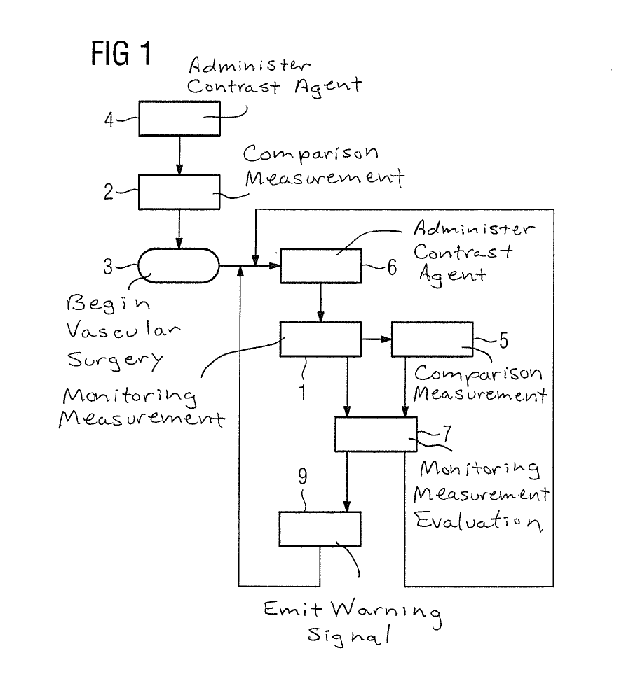

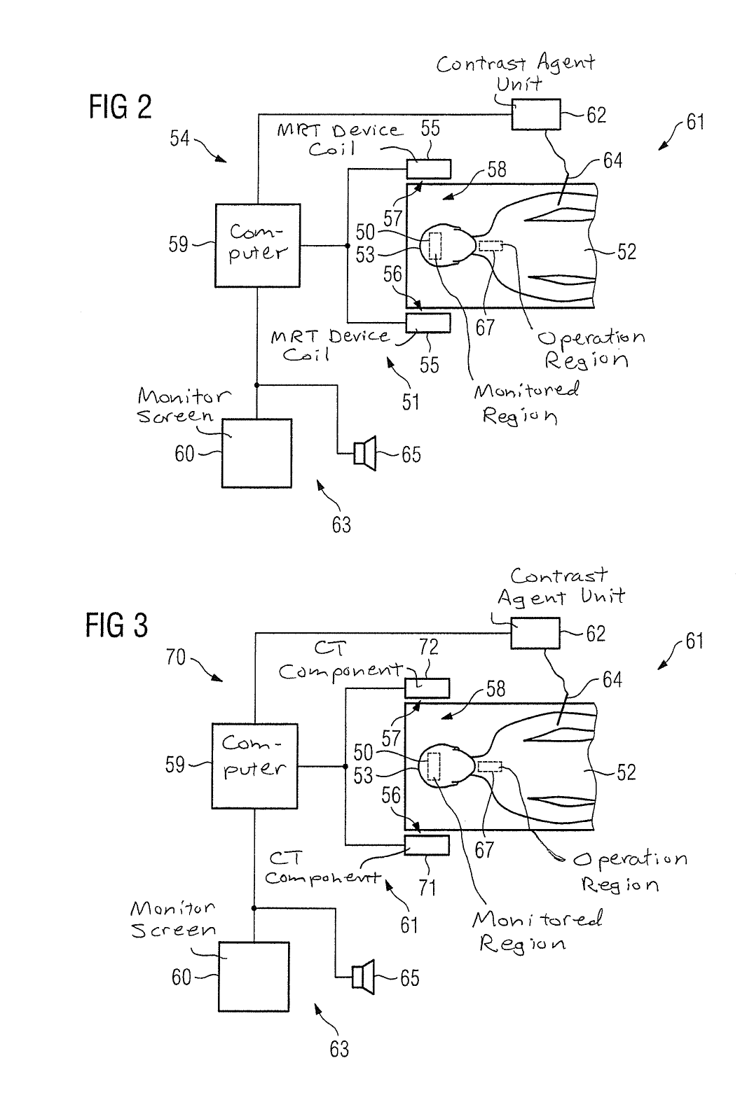

A monitoring method according to the invention for monitoring a medical procedure involving a vascular surgery operation is shown in FIG. 1. The vascular surgery operation is monitored by at least one monitoring measurement 1 in a monitoring region 50, wherein the monitoring measurement 1 takes place by means of a medical imaging device 51 of a monitoring device 61 (FIG. 2). The monitoring region 50 has at least one body part 53 of a patient 52 whose supply depends on a vessel to be operated on, for example the brain given an operation on a cervical artery and/or a foot given an operation on an artery in a leg. In the present exemplary embodiment, the monitoring region 50 is limited to the brain of a patient.

The medical imaging device 51 is formed by a magnetic resonance tomography device 54 in FIG. 2. The magnetic resonance tomography device 54 has two magnetic coils 55 that are arranged on opposite sides 56, 57 of an acquisition region 58 to acquire the patient 52. The two magnetic coils 55 are provided to generate a magnetic field and to detect measurement signals. Furthermore, the magnetic resonance tomography device 54 has a computer 59 that is provided to activate the two magnetic coils 55 and additional units and/or components of the magnetic resonance tomography device 54. The computer 59 is additionally provided for a data evaluation in the operation of the magnetic resonance tomography device 54. The magnetic resonance tomography device 54 has a monitor output 60 for an optical output of measurement results and information. The magnetic resonance tomography device 54 is additionally designed to be mobile so that it can be used for different monitoring regions 50 at patients in a vascular surgery operation.

The monitoring device 61 from FIG. 2 additionally has a contrast agent unit 62, an alarm output unit 63 and a computer 59. The contrast agent unit 62 is provided to administer a contrast agent before the monitoring measurement 1 in order to achieve a contrast increase, for example in an angiography imaging method. For this the contrast agent unit 62 has an insertion element 64 (formed by a hypodermic needle) by means of which the contrast agent can be introduced into the patient 52. The alarm output unit 63 is provided for an optical and acoustic output of a warning signal and for this comprises the monitor output 60 and an acoustic output element 65.

In the operation of the monitoring device 61, the computer 59 is configured for evaluation of data of the magnetic resonance tomography (MRT) device 54, and for a comparison of monitoring variables of the monitoring measurement 1 with at least one reference variable of a comparison measurement 2, 5. The computer 59 can be formed by the control computer of the magnetic resonance tomography device 54 so that additional components and costs can advantageously be spared. The computer 59 is connected in a data exchange with all units and components of the magnetic resonance tomography device 54 and the monitoring device 61 via a data line (not shown in detail). In operation of the monitoring device 61, a control of the contrast agent unit 62 and the alarm output unit 65 likewise ensues by means of the computer 59.

At the beginning of the monitoring method (FIG. 1), a contrast agent administration 4 ensues initially and a first comparison measurement 2 subsequently ensues by means of the magnetic resonance tomography device 54 that is conducted before a beginning 3 of the vascular surgery operation on the patient 52. The first comparison measurement 2 ensues by means of a perfusion imaging method with a contrast agent or by means of an angiography imaging method with a contrast agent. Insofar as the first comparison measurement 2 ensues by means of a diffusion imaging method and/or a susceptibility-weighted imaging method and/or a T2*-weighted imaging method, contrast agent administration 4 can be omitted and the monitoring method begins with the first comparison measurement 2. In addition to the perfusion imaging method or the angiography imaging method with a contrast agent, the comparison measurement 2 can also ensue without contrast agent via the perfusion imaging method—for example by means of ASL (Arterial Spin Labeling)—or the angiography imaging method—for example by means of ToF Angiography, such that the method step of the contrast agent administration 4 can also hereby be omitted. The first comparison measurement 2 likewise ensues in the monitoring region 50 and depicts the brain (whose supply is dependent on the vessel to be operated on, here the cervical artery) in a normal supply.

For the first comparison measurement 2 and for additional comparison measurements 5 in the further course of the monitoring method, and also for the monitoring measurements 1 themselves, it is exclusively the body part 53 of the patient 52 whose supply is dependent on the vessel to be operated on (the head of the patient in FIG. 2) that is positioned in the acquisition region 68 of the magnetic resonance tomography device 54 that is formed by a detector region. The head of the patient 52 is hereby located between the two magnetic coils 55 of the magnetic resonance tomography device 54. An operation (surgery) region 67 affected by the vascular surgery operation is thereby positioned outside of the acquisition region 58 of the magnetic resonance tomography device 54 and therefore is freely accessible to a physician. A possible negative effect on the vascular surgery operation, for example by a magnetic field of the magnetic resonance tomography device 54, is additionally prevented. The operation region 67 is also formed separate from the monitoring region 50.

The vascular surgery operation begins after the first comparison measurement 2. An additional administration 6 of contrast agent initially ensues with the beginning 3 of the vascular surgery operation and a first monitoring measurement 1 subsequently ensues by means of the magnetic resonance tomography device 54 (FIG. 1). The first monitoring measurement 1 ensues by means of a perfusion imaging method with a contrast agent or by means of an angiography imaging method with a contrast agent. Insofar as the first monitoring measurement 1 by means of a diffusion imaging method leads to a depiction of a diffusion flow in the brain and/or a susceptibility-weighted imaging method leads to a depiction of aneurysms or air embolisms in the brain, contrast agent administration 6 can be omitted and the first monitoring measurement 1 of the monitoring region 50 starts with the beginning 3 of the vascular surgery operation. In addition to the perfusion imaging method or the angiography imaging method with a contrast agent, the monitoring measurement 1 can proceed without contrast agent via the perfusion imaging method—for example by means of ASL (Arterial Spin Labeling)—or the angiography imaging method—for example by means of ToF Angiography.

A further comparison measurement 5 additionally takes place at the same time as the monitoring measurement 1, for example in that both hemispheres of the brain are acquired in a measurement, wherein only one of the two brain hemispheres is dependent on the supply from the vessel affected by the surgical operation. The further comparison measurement 5 and the monitoring measurement 1 are thus acquired in one measurement step. The additional comparison measurement 5 thus also additionally takes place at the same time as the vascular surgery operation. Given multiple monitoring measurements 1 the last acquired monitoring measurement 1 can also form the comparison measurement 5 for a current monitoring measurement 1 insofar as no danger of damage to the brain was detected in the last acquired monitoring measurement 1.

After the monitoring measurement 1 and the comparison measurements 2, 5, the acquired data are relayed to the computer 59. An evaluation 7 of the data of the monitoring measurement 1 and the comparison measurements 2, 5 takes place in the computer 59, wherein for this one or more reference variables are determined from the comparison measurements 2, 5. For example, the reference variables indicate a vessel pattern of perfused vessels of the corresponding hemisphere of the brain and/or a diffusion value in the vessels etc. One or more monitoring variables that are comparable with the reference variable of the comparison measurements 2, 5 are determined from the data of the monitoring measurement 1. For example, the monitoring variable of the monitoring measurement 1 likewise indicates a vessel pattern of perfused vessels of the corresponding hemisphere of the brain and/or a diffusion value in the vessels etc. The monitoring variable and the reference variable are hereby formed by an identical variable.

The monitoring variable is thereupon compared with the reference variable within the evaluation 7 in the computer 59. For this a tolerance range around the reference variable is established within which a deviation of the monitoring variable from the reference variable is still tolerable. Alternatively, the comparison of the monitoring variable with the reference variable can also take place manually. Given the simultaneous measurement of the monitoring measurement 1 and the comparison measurement 5 in that both hemispheres of the brain are acquired by means of the magnetic resonance tomography device 54, both sides or, respectively, both hemispheres of the brain are compared with one another in the evaluation 7 by the computer 59. The two hemispheres of the brain hereby respectively form essentially the mirror image of the respective other brain hemisphere relative to the midsagittal plane. In a susceptibility measurement, the reference variable can be formed by an essentially equivalent value of the monitoring variable. Changes to a perfusion in the monitoring region 50 (caused by air bubbles in the vessels, for example) cause local spikes in a curve of the susceptibility, such that a change to the susceptibility can be viewed as a danger of damage to the brain.

As soon as the deviation of the monitoring variable relative to the reference variable lies outside of the tolerance range, this is classified by the computer 59 as a danger of damage to the body part 53 (here the brain) dependent on the supply of the vessel being operated on. Furthermore, a danger of damage to the brain is established by the computer 59 if, for example, an asymmetry in the supply of the individual blood vessels between the two brain hemispheres is established. A danger of damage can additionally be detected if the monitoring measurement 1 and the comparison measurement 2, 5 ensue by means of an angiography imaging method and the two measurement results are subtracted from one another. Insofar as a difference in the vessel pattern thereby results (for example due to missing vessel branches that were present in the comparison measurement), an undersupply of the brain is assumed and a danger of damage to the brain is established.

If a danger of damage to the brain is determined to be present by the computer 59, a warning signal is generated and this is conducted to the alarm output unit 63. A warning signal output 9 ensues by means of the monitor screen 60 and the acoustic output element 65 so that the physician is notified of the danger immediately after its appearance. The physician can thereupon introduce possible countermeasures that counteract the damage to the brain, for example. However, a decision and/or assessment by the physician takes place as to whether a danger potential for damage to the brain exists, and possibly as to a severity of the damage.

If no deviation between the monitoring variable and the reference variable is established in the evaluation 7, a new monitoring measurement 1 is started with prior contrast agent administration 6 (insofar as this is required). Given the presence of a danger of damage to the brain, a new monitoring measurement 1 is also likewise started in parallel with the warning signal output 9 so that a curve of the supply of the brain can be displayed to the physician. The individual monitoring measurements 1 with subsequent evaluation 7 are quickly switched in series with minimal time intervals so that a continuous monitoring via multiple monitoring measurements 1 in parallel with the vascular surgery operation is achieved. A continuous monitoring and/or a continuous observation of the brain are additionally depicted by means of the monitor screen 60 so that a supply and/or a state of the supply of the brain can be visually comprehended by the physician at any time.

In FIG. 3 the monitoring method according to the invention is presented using a further embodiment of the monitoring device 61. The medical imaging device 51 is hereby formed by a computer tomography device 70. Instead of the two magnetic coils, here two computer tomography (CT) units 71 (radiation source and radiation detector) are arranged. A mode of operation of the monitoring method by means of the computer tomography device is limited to an angiography imaging method. A further mode of operation of the monitoring method and the monitoring device 51 thereby corresponds to the description associated to FIGS. 1 and 2.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for monitoring a vascular surgery operation wherein vascular surgery is performed at a vascular surgery site in the body of a patient, comprising the steps of:
with a medical imaging device, monitoring a region of the body of the patient that does not encompass said vascular surgery site, which is at risk of damage due to said vascular surgery, by obtaining an image of said region; and
obtaining said image of said region in a monitoring measurement that takes place simultaneously with said vascular surgery.

2. A monitoring method as claimed in claim 1 comprising making multiple monitoring measurements, each including an image of said region, continuously in succession during said vascular surgery.

3. A monitoring method as claimed in claim 1 comprising obtaining at least one reference variable and making a comparison between information contained in said image and said at least one reference variable to detect an impermissible deviation of said information from said reference variable.

4. A monitoring method as claimed in claim 3 comprising making said comparison at a time selected from the group consisting of before making said monitoring measurement and before beginning said vascular surgery.

5. A monitoring method as claimed in claim 3 comprising making said comparison substantially in parallel with said monitoring measurement or said vascular surgery.

6. A monitoring method as claimed in claim 3 comprising obtaining at least one monitoring variable, as said information, from said monitoring measurement.

7. A monitoring method as claimed in claim 3 comprising emitting a warning signal if said deviation exceeds a predetermined tolerance range with respect to said reference variable.

8. A monitoring method as claimed in claim 1 comprising obtaining said image by an imaging modality selected from the group consisting of perfusion imaging, angiography imaging, diffusion imaging, susceptibility-weighted imaging and T2*-weighted imaging.

9. A monitoring method as claimed in claim 1 comprising administering a contrast agent to the patient before said monitoring measurement, and generating said image in said monitoring measurement using said contrast agent.

10. A device for monitoring a vascular surgery operation wherein vascular surgery is performed at a vascular surgery site in the body of a patient, comprising:
a medical imaging device that monitors a region of the body of the patient that does not encompass said vascular surgery site, which is at risk of damage due to said vascular surgery, by obtaining an image of said region;
said medical imaging device being configured to obtain said image of said region in a monitoring measurement that takes place simultaneously with said vascular surgery; and
a computer evaluation device configured to evaluate said monitoring measurement simultaneously with said vascular surgery.

11. A monitoring device as claimed in claim 10 comprising wherein said medical imaging device is configured to make multiple monitoring measurements, each including an image of said region, continuously in succession during said vascular surgery.

12. A monitoring device as claimed in claim 10 wherein said evaluation unit is configured to obtain at least one reference variable and making a comparison between information contained in said image and said at least one reference variable to detect an impermissible deviation of said information from said reference variable.

13. A monitoring device as claimed in claim 12 wherein said evaluation unit is configured to make said comparison at a time selected from the group consisting of before making said monitoring measurement and before beginning said vascular surgery.

14. A monitoring device as claimed in claim 12 wherein said evaluation unit is configured to make said comparison substantially in parallel with said monitoring measurement or said vascular surgery.

15. A monitoring device as claimed in claim 12 wherein said evaluation unit is configured to obtain at least one monitoring variable, as said information, from said monitoring measurement.

16. A monitoring device as claimed in claim 12 wherein said evaluation unit is configured to emit a warning signal if said deviation exceeds a predetermined tolerance range with respect to said reference variable.

17. A monitoring device as claimed in claim 10 wherein said medical imaging device is configured to operate to perform an imaging modality selected from the group consisting of perfusion imaging, angiography imaging, diffusion imaging, susceptibility-weighted imaging and T2*-weighted imaging.

18. A monitoring device as claimed in claim 10 comprising administering a contrast agent unit configured to administer a contrast agent to the patient before said monitoring measurement, and wherein said imaging device is configured to generate said image in said monitoring measurement using said contrast agent.
